(19) United States
(12) Patent Application Publication
(10) Pub. No.: US 2006/0106119 A1
(43) Pub. Date: May 18, 2006

(54) Novel integration for CO and H2 recovery in gas to liquid processes

(76) Inventors: Chang-Jie Guo, Exton, PA (US); Paul Wentink, Feucherolles (FR)

Correspondence Address: AIR LIQUIDE, 2700 POST OAK BOULEVARD, SUITE 1800, HOUSTON, TX, 77056, US

(21) Appl. No.: 11/255,461

(22) Filed: Oct. 21, 2005

Related U.S. Application Data
(63) Continuation-in-part of application No. 10/957,457, filed on Oct. 1, 2004.
(60) Provisional application No. 60/535,786, filed on Jan. 12, 2004.

(51) Int. Cl. C07B 63/02; C07C 27/26

(52) U.S. Cl. 518/726

(57) ABSTRACT

Methods for integrating a syngas production unit with a gas to liquid (GTL) system, and a power generation unit to efficiently use hydrogen and carbon monoxide contained in the syngas produced from methane-containing hydrocarbon feedstock (e.g. natural gas). Membrane separation and other separation technologies are used to separate components of an off-gas stream from the GTL system and associated down stream hydroprocessing unit, and recycle the stream components advantageously to the process, or use them in utility generation units. A hydrogen-recovery membrane unit and a CO-recovery unit are utilized also to produce a syngas feed stock to the GTL system with an H₂/CO ratio favorable for the production on synthetic liquid hydrocarbons. In one embodiment, pure hydrogen is also produced in a PSA unit, whose feed stream enriched in H₂ by a membrane to provide hydrogen needed for liquid product hydroprocessing systems.

### CROSS REFERENCE TO RELATED APPLICATIONS

This application is a CIP of U.S. application Ser. No. 10/957,457, titled “Novel Integration of Gasification Hydrocarbon Synthesis Unit”, filed Oct. 1, 2004, which is related to and claims the benefit of U.S. Provisional Application No. 60/535,786, filed Jan. 12, 2004. The entire contents of both applications are incorporated herein by reference.

### BACKGROUND

This invention relates to the integration of Gas to Liquid (GTL) system and its associated product hydroprocessing units with syngas production units, and power generation units through the use of gas separation methods that include membrane permeation, adsorption, and absorption to effectively utilize H₂, and CO contained in raw material feedstock. The advantages are increased synthetic product production per unit of feedstock and full utilization of stream components as chemical feedstocks or power generation fuel. The integration of these operations also significantly reduces number of separation units required.

Syngas (a mixture of CO and H₂) is produced from a variety of feedstocks ranging from heavy oil, coal to light methane-containing gases. As world crude prices continue to rise, the conversion of gases containing primarily methane, such as natural gas (especially those in regions isolated from major markets), to synthetic hydrocarbon products becomes more attractive. A potentially economical option is to use methane-containing hydrocarbon feedstocks, such as natural gas, to generate a syngas, while also generating utility products (power and steam). These products are then used by GTL systems, hydrocracker units, or sold on the open market. GTL systems typically use a Fisher-Tropsch reaction to convert the syngas to synthetic hydrocarbons, such as ultra-clean transportation fuels, methanol, and naphtha.

Of particular interest is the conversion of natural gas to syngas by processes such as steam methane reforming (SMR), combined methane reforming (CMR), autothermal reforming (ATR), or partial oxidation (POX). These processes can produce syngas with H₂/CO ratios of about 3-6, 2.5-4, 1.9-2.6, and 1-1.9, respectively. The syngas is used in many industrial chemical production applications, including gas to liquid (GTL) processes. A GTL plant may comprise syngas conversion systems, such as Fischer-Tropsch (F-T) reactors, liquid/vapor separation systems, and/or other equipment.

A given H₂/CO ratio is usually required of syngas that is utilized as feedstock to F-T based GTL processes. For instance, one F-T process requires a syngas with a H₂/CO ratio of about 2.0. Either adding an H₂-rich stream to the syngas or removing H₂ from the syngas, depending on the syngas generating process as mentioned above, can adjust the H₂/CO ratio to the desired levels. Furthermore, since the syngas conversion in the F-T reactors is usually much lower than 100%, the gaseous stream, after being separated from liquid, is mostly recycled back to the F-T reactors. To avoid build-up of inert components in the reactor system (such as Ar, CO₂, and C₁-C₅ hydrocarbons) a portion of the recycle gaseous stream need to be purged. The purge results in loss of valuable syngas components, CO and H₂. It is desirable to develop processes that efficiently use all of the contained H₂, CO, and energy in the feedstocks while supplying syngas with the required H₂/CO ratio to hydrocarbon synthesis units.

It is further desirable to minimize the overall energy consumed by the syngas/GTL processes. Methods of minimizing energy consumption include using undesirable stream components (i.e.: C₁-C₅ hydrocarbons) as fuel to burn in furnaces or power generators, while minimizing the amount of mechanical compression or pumping of process streams. Thus, processes that maximize the use of all stream components while minimizing the compression of large-volume streams are desirable. F-T reactor products are usually routed to hydrotreating/hydrocracking units where the synthetic hydrocarbons are further modified to produce desired final products, such as diesel. Hydrotreators (hydrotreating reactors) treat the synthetic hydrocarbon feedstock catalytically in the presence of an excess of hydrogen to modify the feedstock to the desired chemical structure. However, it is difficult to maintain the high levels of hydrogen in the hydrotreator, due to a buildup of inert gases in the system. To remove the inert gases, a portion of the recycle gas is normally purged to continuously remove inert gases from the hydrotreating system. The hydrogen required by the reactions is supplied through a make-up stream that usually has a high H₂ content. The more make-up stream is used, and the more recycle gas is purged, the higher the H₂ partial pressure in the hydrotreating reactors. Since the recycle gas is high in hydrogen content, purging will result in significant hydrogen losses to the process. Thus, it is desirable to reject non-hydrogen components in the purge-gas stream while recapturing the contained hydrogen. It is also desirable to extract value, such as the heating value, from the non-hydrogen components of the purge stream.

There are several important factors to the efficient conversion of methane-containing feedstocks to high value fuels, chemicals, and power. It is particularly desirable to:
Minimize the loss of CO and H₂ in the combined syngas/F-T/hydrotreating processes;
Reject undesirable components from the GTL process while capturing and recycling the valuable components of the feedstock such as H₂ and CO;
Maximize the use of contained energy in feedstock by converting undesirable components to energy;
Minimize the energy consumed compressing process streams;
Provide high purity make-up H₂ for hydroprocessing units; and
Reject light hydrocarbons and capture the H₂ content of hydroprocessing purge streams.

Thus, it is desirable to develop processes that maximize production of high value products while minimizing the loss of valuable feedstock components and energy consumption across the entire chain of syngas production, GTL conversion, utilities generation, and final product production.

### SUMMARY

The present invention is directed to a process that satisfies the need to maximize production of high value products while minimizing the loss of valuable feedstock components and minimizing energy consumption across the chain of syngas production, GTL operations, power and steam generation, and final high quality fuel production. This is accomplished in the present invention by integrating a syngas generation unit, an F-T system, and a utilities generation unit.

According to one embodiment of the invention, a methane-containing feedstock comprising methane is supplied to a syngas production unit where a syngas is made. The syngas is a primary component of a feedstock for the F-T reactors of a GTL system. The GTL system produces a mixture of hydrocarbons with other process inert gases. When the heavy hydrocarbons (such as C₆⁺) are separated from light components in a vapor/liquid separator, a GTL off-gas is formed as the gaseous effluent of the separator. A large portion of this off-gas, containing significant amount of unconverted CO and H₂, is directly re-circulated back to the F-T reactors. A portion is separated in an off-gas membrane separator to form an H₂-enriched gas and an H₂-lean/CO-rich gas. The H₂-lean/CO-rich gas is fed to a CO recovery unit to form a CO-enriched gas and a combustible tail gas. The CO-enriched gas is then combined with the syngas stream leaving the syngas production unit to form a CO-enriched syngas. The CO-enriched syngas is in turn combined with the H₂-enriched gas from the off-gas membrane separator to form an H₂-enriched syngas. Next, the H₂-enriched syngas is combined with a second portion of GTL off-gas to form the GTL feedstock with the proper H₂/CO ratio required to produce the desired synthetic hydrocarbon products. Furthermore, the combustible tail gas from the CO recovery unit is sent to a utilities generation unit to produce a utility product such as steam, or electricity.

In other embodiments:
a third portion of GTL off-gas is recycled to the syngas production unit;
a fourth portion of GTL off-gas is fed to the utilities generation unit to generate a utility.

Furthermore, other embodiments allow for the use of syngas-generation unit feedstocks containing relatively high levels of CO₂ by routing the feedstock to a feedstock membrane separator, where the CO₂ content is adjusted and used in an SMR unit to form a SMR syngas, which in turn is used to raise the CO content of the syngas exiting a SMR, ATR, POX, or other type of syngas production unit.

The current invention also provides a method to integrate a syngas production unit, a GTL system, a utilities generation unit, and a hydroprocessing system. In this embodiment, as with the embodiments above, at least a part of the GTL off-gas is directly recycled back to the F-T reactor or the syngas generation section, another part is routed to an H₂-off-gas membrane separator, and yet another part sent optionally to a utilities unit. In this scheme, however, at least a portion of the purge stream from a down stream synthetic product hydroprocessing system is also fed to the membrane unit to recover the H₂ contained in the hydroprocessor off-gas and convert undesirable combustible components into energy.

### BRIEF DESCRIPTION OF THE DRAWINGS

For a further understanding of the nature and objects of the present invention, reference should be made to the following detailed description, taken in conjunction with the accompanying drawings, in which like elements are given the same or analogous reference numbers and wherein:

FIG. 1 is a diagram of one embodiment of the current invention;

FIG. 2 is a diagram of a second embodiment of the current invention;

FIG. 3 is a diagram of a third embodiment of the current invention; and

FIG. 4 is an example mass balance for the embodiment of FIG. 1.

### DESCRIPTION OF PREFERRED EMBODIMENTS

The process of the present invention integrates a chain of processes, including a syngas production unit, an F-T based hydrocarbon synthesis system, and a utilities generation unit, to produce a synthetic hydrocarbon product and power from a methane-containing feedstock while minimizing the losses of valuable feedstock components, such as CO and H₂. Optionally, a hydroprocessing system may be included in the chain to efficiently utilize H₂ in the hydrotreator (or hydrocracker) purge stream. The process utilizes gas separation technologies, such as absorption systems and membrane separators to recover valuable stream components and feed them to the unit where the component can be most effectively utilized. The method provides an increase of about 7 to 10% in F-T Liquid production from a fixed natural gas feed.

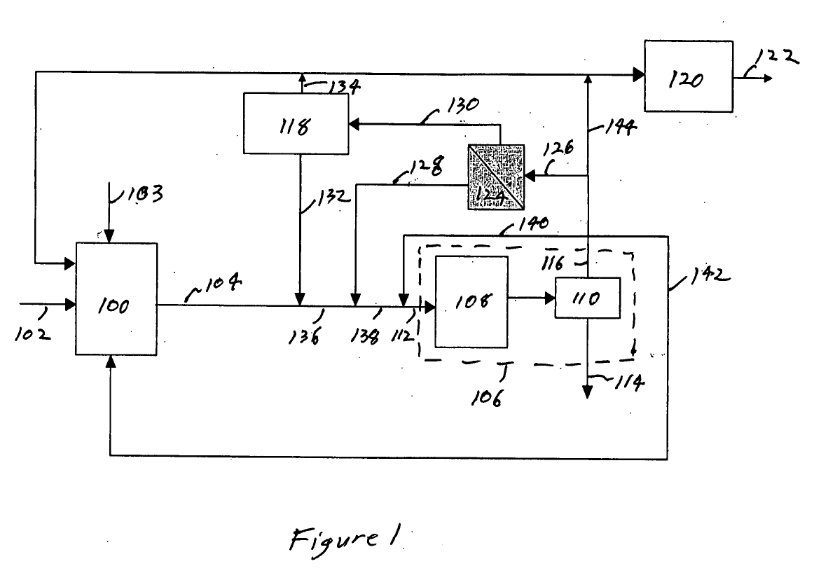

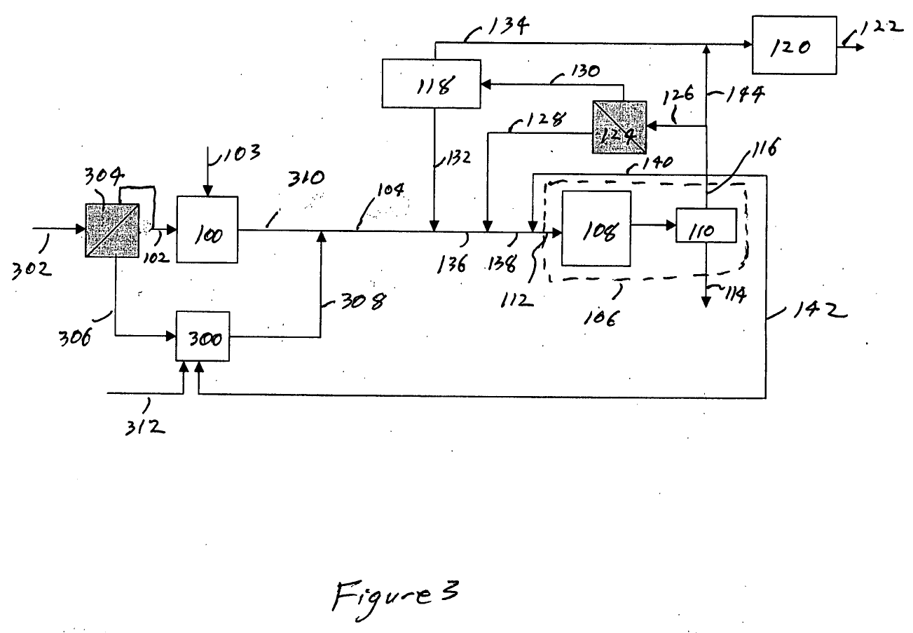

Referring to FIGS. 1 to 3, syngas production unit 100 refers to any process known to one of ordinary skill in the art to convert a hydrocarbon feedstock comprising methane into a syngas comprising primarily carbon monoxide (CO), hydrogen (H₂), and carbon dioxide (CO₂). The syngas production unit 100 preferably uses steam methane reforming (SMR), combined methane reforming (CMR), autothermal reforming (ATR), or partial oxidation (POX) for the conversion process. The methane-containing feedstock 102 contains significant quantities of methane, and may be natural gas. In one embodiment, preferred processes utilize an oxygen-containing stream 103 to produce a syngas 104 with a H₂/CO ratio of greater than about 1.0, more preferably greater than about 1.5 and even more preferably in a range of about 1.8 to 2.2. However, the process is also applicable to processes using any H₂/CO ratio. Furthermore, the syngas 104 contains greater than about 40 mole percent (mol %) H₂, greater than 50 mol % H₂, or in a range of about 55 to 65 mol % H₂. These ranges are subject to change with changing methane-containing feedstock. The oxygen-containing stream 103 is preferably a substantially pure oxygen stream for ATR and POX units. For units such as an SMR unit of FIG. 3, the methane-containing feedstock 102 is preferably reacted with an H₂O stream 312 to produce a SMR syngas 308).

Referring again to FIGS. 1 to 3, a GTL system 106 is any process known to one of ordinary skill in the art for converting a syngas into synthetic liquid hydrocarbon products. Typical processes are, but are not limited to, Fischer-Tropsch (F-T) or chain growth reaction of carbon monoxide and hydrogen on the surface of a heterogeneous catalyst. GTL systems 106 may comprise various sub-parts, such as a gas to liquid reaction zone 108, and a liquid/vapor separation zone 110. A GTL feedstock 112, comprising syngas 104 is converted to a synthetic hydrocarbon product 114 by the reaction of the GTL feedstock 112 in the GTL system 106. The synthetic hydrocarbon product 114 is separated as a liquid from the unreacted H₂, CO, inerts, and/or other unreacted syngas components in the liquid/vapor separation zone 110. The unreacted H₂, CO, inerts, and other unreacted syngas components are removed from the liquid/vapor separation zone as a GTL off-gas stream 116. Because there is a significant amount H₂, CO, and other valuable components in the GTL off-gas stream 116, recycle and recovery of this stream greatly improves system efficiency.

Still referring to FIGS. 1 to 3, a CO recovery unit 118 is any process known to one of ordinary skill in the art where CO is selectively extracted (via adsorption, absorption, or other means) over other components of a feed to the unit. Preferred CO recovery units include vacuum swing adsorption, pressure swing adsorption, or any other devices that separate CO from N₂, CH₄, Ar, and C₁-C₅ hydrocarbons. A CO-rich product and a CO-lean waste gas are produced from the CO recovery unit. The CO-rich stream is recycled back to the F-T reactor feed while the CO-lean stream is sent to a utilities generation unit 120 as a fuel.

Still referring to FIGS. 1 to 3, a utilities generation unit 120 is a process or unit that produces a utility product 122. As used herein, a utility product is any product produced and used as a power or heat source. The utility product is preferably hot water, steam, or electricity. The utilities generation unit can be any process known to one skilled in the art, such as a simple boiler that converts a fuel stream into steam, which in turn is used as a power source. Preferred utilities generation units include co-generation units, and combined cycle units. Combined cycle units burn a fuel stream and use both gas and steam turbine cycles in a single plant to produce electricity and steam with high conversion efficiencies and low emissions.

Again referring to FIGS. 1 to 3, an off-gas membrane separator 124 is any membrane separation device or membrane materials known to one skilled in the art effective for separation of H₂ by preferential permeation of H₂ over CO, CO₂, or any other ordinary gases encountered in GTL off-gas 116. A preferred membrane is permeable primarily to H₂, passing only small amounts of CO₂. Any type of construction for membrane separators may be used, although hollow-fiber type is preferred for its compactness and high separation efficiency. Membranes may be and suitable asymmetric membranes, composite membranes, or mixed matrix membranes. Representative membrane materials include polysulfone, polyether sulfone, polyamide, polyimide, polyetherimide, polyesters, polycarbonates, copolycarbonate esters, polyethers, polyetherketones, polyvinylidene fluoride, polybenzimidazoles, polybenzoxazoles, cellulosic derivatives, polyazoaromatics, poly (2,6-dimethylphenylene oxide), polyarylene oxide, polyureas, polyurethanes, polyhydrazides, polyazomethines, cellulose acetates, cellulose nitrates, ethyl cellulose, brominated poly (xylylene oxide), sulfonated poly (xylylene oxide), polyquinoxaline, polyamideimides, polyamide esters, blends thereof, copolymers thereof, substituted materials thereof and the like. Polyimide polymer membranes may include:
(a) Type I polyimides and polyimide polymer blends as described in co-pending application Ser. No. 10/642,407, titled “Polyimide Blends for Gas Separation Membranes”, filed Aug. 15, 2003, the entire disclosure of which is hereby incorporated by reference;
(b) polyimide/polyimide-amide and polyimide/polyamide polymer blends as described in co-pending application Ser. No. 11/036,569, titled, “Novel Separation Membrane Made From Blends of Polyimide With Polyamide or Polyimide-Amide Polymers”, filed Jan. 14, 2005, the entire disclosure of which is hereby incorporated by reference; and
(c) annealed polyimide polymers as described in co-pending application Ser. No. 11/070,041, titled, “Improved Separation Membrane by Controlled Annealing of Polyimide Polymers”, filed Mar. 2, 2005, the entire disclosure of which is hereby incorporated by reference.

Furthermore, the membranes may be mixed matrix membranes, such as mixed matrix membranes as described in co-pending application 11/091,682, titled, “Novell Polyimide Based Mixed Matrix Membranes”, filed Mar. 28, 2005, electrostabilized mixed matrix membranes as described in co-pending application Ser. No. 11/091,619, titled, “Novel Method Of Making Mixed Matrix Membranes Using Electrostatically Stabilized Suspensions”, filed Mar. 28, 2005, and mixed matrix membranes with washed molecular sieve particles as described in co-pending application Ser. No. 11/091,156, titled, “Novell Method For Forming A Mixed Matrix Composite Membrane Using Washed Molecular Sieve Particles”, filed Mar. 28, 2005. The entire disclosures of the applications mentioned above are hereby incorporated by reference.

The membrane materials described above should not be considered limiting since any material that can be fabricated into an anisotropic membrane may be able to be employed for the separation tasks here. These may include H₂-selective membrane made of metal (Pd) or metal alloy (Pd—Cu) or inorganic materials (such as ceramic).

The membrane unit extracts greater than 50% and preferably, greater than 85% of the H₂ in the off-gas as a hydrogen rich permeate stream at a pressure significantly lower than the membrane feed. The H₂ stream, which is relatively small, is re-compressed and fed into the F-T reactor feed stream as needed. The membrane residue stream that is lean in H₂ but rich in CO, and still near off-gas pressure is sent to the CO recovery unit.

Referring to FIG. 1, one preferred embodiment of the current process integrates a syngas production unit 100, a GTL system 106, and a utilities generation unit 120. In this embodiment, a GTL feedstock 112 is fed to a GTL system 106 where a synthetic hydrocarbon product 114 is produced and a GTL off-gas 116 is originated. A major portion of the GTL offgas 116 is recirculated. A first portion of GTL off-gas 126 is routed to an off-gas membrane separator 124 where it is separated into an H₂-enriched gas 128 and an H₂-lean/CO-rich gas 130. The H₂-lean/CO-rich gas 130 is routed to a CO recovery unit 118, where it is separated into a CO-enriched gas 132 and a CO-lean gas 134. The CO-enriched gas 132 is combined with a syngas 104 to form a first CO-enriched syngas 136.

The CO-lean gas 134 is routed back to the syngas production unit 100 for recycle as the process allows, and/or to the utilities generation unit 120 for burning as a fuel to produce a utility product 122. The CO-lean gas 134 contains CO, CO₂, some hydrogen, and other volatile hydrocarbons. This stream makes a suitable fuel, particularly for combustion in the utilities generation unit 120.

The H₂-enriched gas 128 is combined with the first CO-enriched syngas 136 to form an H₂ enriched syngas 138. A second portion of GTL off-gas 140 is combined with the H₂-enriched syngas 138 to form the previously mentioned GTL feedstock 112 with the proper H₂/CO ratio to produce the desired synthetic hydrocarbon product 114. A third portion of GTL off-gas 142 is routed back to the syngas production unit 100 for recycle as the process allows. Optionally, any excess GTL off-gas can be used by routing a fourth portion of GTL off-gas 144 to the utilities generation unit 120 for producing a utility product 122. The H₂/CO ratio of the GTL feedstock 112, as well as the overall process economics, can be optimized by adjusting the partition of the GTL off-gas 116 into the first portion of GTL off-gas 126, second portion of GTL off-gas 140, and third portion of GTL off-gas 142 respectively.

The embodiment shown in FIG. 1 provides for efficient use of the H₂ and CO contained in the syngas 104 by either recycling the H₂ and CO components or extracting the contained energy in the GTL off-gas 116. A typical example of the net recovery (expressed as normal cubic meters of gas per barrel of product) from an off-gas separation stream is summarized in Table 1.

TABLE 1
CO and H₂ recovery from GTL off-gas
Stream Composition (mol %)

| | GTL Off- | Recycled | | |
|---|---|---|---|---|
| Components | gas | H₂ | Recycled CO | Fuel gas |
| CO | 28.56% | 0.00% | 96.66% | 6.24% |
| CO₂ | 7.94% | 0.00% | 0.00% | 17.35% |
| Hydrogen | 30.60% | 94.22% | 1.72% | 9.02% |
| H₂O | 1.63% | 0.00% | 0.00% | 3.56% |
| Nitrogen | 2.11% | 0.38% | 0.00% | 4.37% |
| Methane | 22.29% | 0.00% | 1.68% | 47.71% |
| Ethane | 0.34% | 0.00% | 0.00% | 0.74% |
| Propane | 1.14% | 0.00% | 0.00% | 2.49% |
| n-Butane | 1.41% | 0.00% | 0.00% | 3.09% |
| n-Pentane | 0.84% | 0.00% | 0.00% | 1.83% |
| n-Hexane | 0.57% | 0.00% | 0.00% | 1.25% |
| Ethylene | 0.15% | 0.00% | 0.00% | 0.32% |
| Propylene | 0.28% | 0.00% | 0.00% | 0.62% |
| Argon | 2.13% | 5.39% | 0.00% | 1.39% |
| | 100.00% | 100.00% | 100.00% | 100.00% |
| Nm3/barrel | 5.739 | 2.552 | 1.496 | 2.691 |
| products | | | | |

This recovery operation, when considering a 35,000 bpd F-T plant with its syngas unit will effectively produce 38,000 bpd additional barrels. When considering a grassroots application, the investment will be paid for by the 7-10% reduction in required syngas generation capacity leaving the reduced feed consumption as operation advantage.

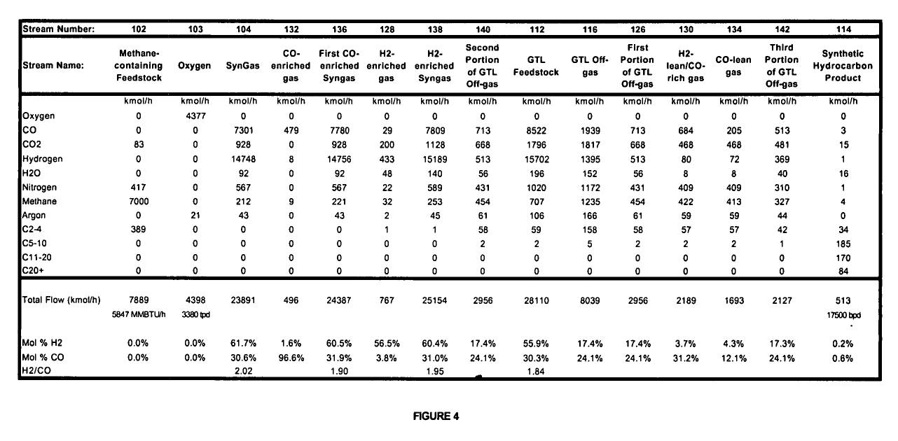

The GTL feedstock 112 is formed with an effective H₂/CO ratio to produce the desired synthetic hydrocarbon product 114. In one preferred embodiment, the effective H₂/CO ratio for the GTL feedstock 112 is greater than about 1.0, more preferably greater than about 1.5 and even more preferably in a range of about 1.8 to 2.2. One skilled in the art can determine an effective flow rate for the H₂-enriched gas 128 that must be combined with the first CO-enriched syngas 136 to achieve the effective H₂/CO ratio based on mass balance simulations without undue experimentation. A mass balance of one example embodiment according to FIG. 1 for a GTL plant producing 175,000 barrels per day (bpd) of synthetic hydrocarbon product 114 is shown in FIG. 4.

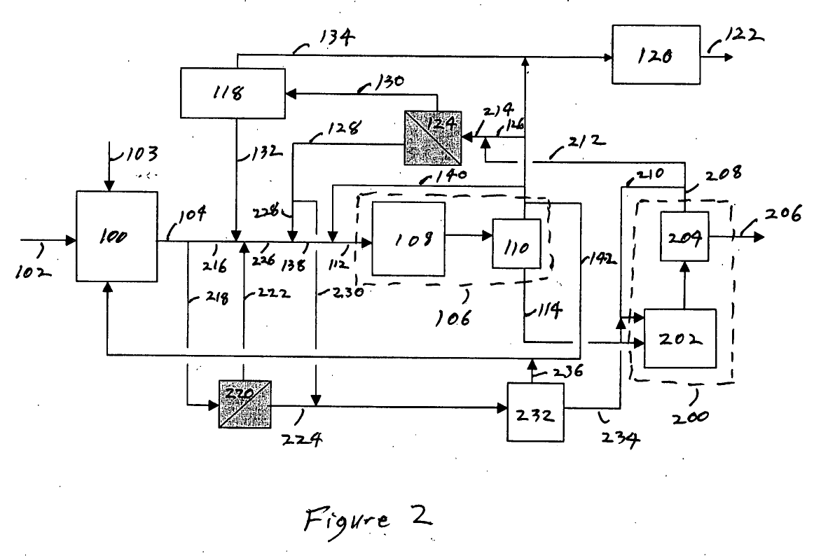

Referring to FIG. 2, one preferred embodiment of the current process integrates a syngas production unit 100, a GTL system 106, a utilities generation unit 120, and a synthetic product hydroprocessing system 200. In this preferred embodiment, which is similar to FIG. 1, at least a part of the GTL off-gas 116 is directly recycled back to the reactor or the syngas generation section, another part is routed to an H₂-off-gas membrane separator 124, and yet another part is optionally sent to a utilities unit 120. In this scheme, however, at least a portion of the purge stream from a down stream synthetic product hydroprocessing system 200 is also fed to the membrane unit.

A synthetic product hydroprocessing system 200 preferably comprises a hydroprocessor 202 and a hydroprocessor liquid/vapor separator 204. The hydroprocessor 202 is preferably a hydrotreator or hydrocracker unit. These units operate under excess H₂ presence to catalytically improve quality of their feedstock, as is well known to those skilled in the art. The hydroprocessor 202 utilizes high concentrations of hydrogen to modify the synthetic hydrocarbon product 114 to produce the desired hydroprocessor product 206 with similar characteristics to conventional refinery products, such as liquid fuel. The hydroprocessor liquid/vapor separator 204 allows the process to separate the hydroprocessor product 206 from the vapor, forming a hydroprocessor off-gas 208. Because the hydroprocessor off-gas 208 still contains significant quantities of H₂, a first portion of hydroprocessor purge 210 is recycled directly back to the hydroprocessor 202. However, because inerts build up in the hydroprocessing system 200, a second portion of hydroprocessor off-gas 212 must be removed from the system to prevent inert gas buildup in the system. Integration of the hydroprocessing system 200 with the GTL system 106 allows for optimum utilization of H₂ contained in the hydroprocessor off-gas 212 and avoids a net purge. The recovered H₂ is used in the GTL system 106 to adjust the H₂/CO ratio of the GTL system feedstock 112.

A GTL feedstock 112 is fed to a GTL system 106 where a synthetic hydrocarbon product 114 is produced and a GTL off-gas 116 originates. A first portion of GTL off-gas 126 and the second portion of hydroprocessor purge 212 are combined to form an off-gas/purge stream 214 that is routed to an off-gas membrane separator 124 where it is separated into an H₂-enriched gas 128 and an H₂-lean/CO-rich gas 130. The H₂-lean/CO-rich gas 130 is routed to a CO recovery unit 118, where it is separated into a CO-enriched gas 132 and a CO-lean gas 134. The CO-enriched gas 132 is combined with a first portion of syngas 216 to form a first CO-enriched syngas 136. The CO-lean gas 134 is routed back to the utilities generation unit 120 to produce a utility product 122. The off-gas membrane separator 124 preferably extracts greater than 85% of the H₂ in the combined off-gas/purge stream 214 as the H₂-enriched gas 128. The H₂-enriched gas 128 is the permeate stream of the off-gas membrane separator 124, thus is at a pressure significantly lower than the membrane feed. This stream must be re-compressed to be recycled back to the process, however, because it is a relatively small stream, the compression required by the current method is minimized.

The syngas 104 is divided into the first portion of syngas 216 mentioned above and a second portion of syngas 218. The second portion of syngas 218 is fed to a syngas membrane separator 220 where it is separated into an H₂-lean syngas 222 and an H₂-enriched syngas side stream 224. The syngas membrane separator 220 is any membrane separation device or membrane material known to one skilled in the art effective for separation of H₂ by preferential permeation of H₂ over CO, CO₂, or any other ordinary gases in the syngas 104. Any type of construction for membrane separators may be used, although hollow-fiber type is preferred for its compactness and high separation efficiency. Membranes may be of any of the materials mentioned herein above that are found suitable to this application.

The H₂-lean syngas 222 is combined with the first CO-enriched syngas 136 to form a second CO-enriched syngas 226. Furthermore, the H₂-enriched gas 128 is divided into a first portion of H₂-enriched gas 228 and a second portion of H₂-enriched gas 230. The first portion of H₂-enriched gas 228 is then combined with the second CO-enriched syngas 226 to form an H₂ enriched syngas 138 with an effective amount of H₂ as required further downstream in the GTL feedstock 112. The H₂-enriched syngas 138 is then combined with the second portion of GTL off-gas 140 to form the previously mentioned GTL feedstock 112 with the proper H₂/CO ratio to produce the desired synthetic hydrocarbon product 114. A third portion of GTL off-gas 142 is optionally routed back to the syngas production unit 100 for recycle as the process allows. Optionally, any excess GTL off-gas can be used by routing a fourth portion of GTL off-gas 144 to the utilities generation unit 120 for producing a utility product 122.

The second portion of H₂-enriched gas 230 and the H₂-enriched syngas side stream 224 from the syngas membrane separator 220 are fed to an H₂ PSA unit 232, which produces a high purity H₂ stream 234 and an H₂ PSA tail gas 236. The high purity H₂ stream 234 is then fed to the hydroprocessor 202 as make-up hydrogen along with the first portion of hydroprocessor off-gas 210 to maintain the desired H₂ concentration in the hydroprocessor 202. The H₂ PSA tail gas 236, which is H₂-lean and hydrocarbon-rich, is routed back to the syngas production unit 100 along with the third portion of GTL off-gas 142 as a fuel or feedstock. The high purity H₂ stream 234 of the current invention is preferably greater than about 95 mole percent hydrogen, more preferably greater than about 99 mole percent hydrogen, and even more preferably about 99.99 mole percent hydrogen. The effective feed rate of the second portion of H₂-enriched gas 230 and the H₂-enriched syngas side stream 224 to the H₂ PSA unit 232, and the proper size of the PSA unit can be determined by one skilled in the art to produce the desired flow rate of high purity H₂ without undue experimentation. The syngas membrane unit 220 provides a desired H₂-rich feedgas to the PSA unit 232 to produce high purity H₂ with high efficiency.

In FIG. 3, one preferred embodiment of the current process integrates a syngas production unit 100 (preferably a POX or ATR unit), a GTL system 106, a utilities generation unit 120, and a SMR unit 300. In this arrangement, a CO₂ removal membrane unit is utilized to remove CO₂ in the methane-containing feedstock, usually natural gas, and the CO₂ removed is routed to an SMR unit for additional CO generation. The additional CO produced increases the liquid production rate in down stream F-T reaction stage. This is particularly applicable to cases where natural gas feed stock is characterized by a high CO₂ content, and where a POX/ATR as well as a SMR unit is combined to supply syngas to the F-T liquid plant. In addition to the increased CO generation, this scheme also reduces the steam demand for the SMR. Clearly, the CO₂ removal and utilization scheme can also be integrated with the scheme of FIG. 2 described above.

In the embodiment of FIG. 3, an untreated methane-containing feedstock 302 is fed to a feedstock membrane separator 304. This embodiment operates in the same fashion as described for the embodiment for FIG. 1, except that a feedstock membrane separator 304 separates the untreated methane-containing feedstock 302 into the methane-containing feedstock 102 and a CO₂-enriched feedstock 306. Preferred membrane materials in the feedstock membrane separator 304 remove CO₂ from methane-containing gas, such as natural gas, by selective permeation of CO₂ through the membrane and keep methane on the high-pressure residue side of the membrane. The CO₂ enriched feedstock 306 is fed to the SMR unit 300 where a SMR syngas 308 is produced. The methane-containing feedstock 102, is then fed to the syngas production unit 100 to form an ATR/POX syngas 310. The SMR syngas 308 is combined with the ATR/POX syngas 310 from the syngas production unit 100 to form the syngas 104.

As shown in FIG. 3, the GTL feedstock 112 is fed to a GTL system 106 where a synthetic hydrocarbon product 114 is produced and a GTL off-gas 116 is originated. A first portion of GTL off-gas 126 is routed to an off-gas membrane separator 124 where it is separated into an H₂-enriched gas 128 and an H₂-lean/CO-rich gas 130. The H₂-lean/CO-rich gas 130 is routed to a CO recovery unit 118, where it is separated into a CO-enriched gas 132 and a CO-lean gas 134. The CO-enriched gas 132 is combined with the syngas 104 to form a first CO-enriched syngas 136.

The CO-lean gas 134 is routed to the utilities generation unit 120 as a fuel to produce a utility product 122. The CO-lean gas 134 contains CO, CO₂, some hydrogen, and other volatile hydrocarbons. This stream makes good fuel, particularly for combustion in the utilities generation unit 120.

The H₂-enriched gas 128 is combined with the first CO-enriched syngas 136 to form an H₂ enriched syngas 138. A second portion of GTL off-gas 140 is combined with the H₂-enriched syngas 128 to form the previously mentioned GTL feedstock 112 with the proper H₂/CO ratio to produce the desired synthetic hydrocarbon product 114. A third portion of GTL off-gas 142 is routed back to the SMR unit 300 for recycle as the process allows. Optionally, any excess GTL off-gas can be used by routing a fourth portion of GTL off-gas 144 to the utilities generation unit 120 for producing a utility product 122.

Again referring to FIG. 3, the GTL feedstock 112 is formed with an effective H₂/CO ratio to produce the desired synthetic hydrocarbon product 114. In one preferred embodiment, the effective H₂/CO ratio for the GTL feedstock 112 is the greater than about 1.0, more preferably greater than about 1.5 and even more preferably in a range of about 1.8 to 2.2. However, the current method can also be used with processes of any H₂/CO ratio. One skilled in the art can determine an effective flow rate for the H₂-enriched gas 128 that must be combined with the first CO-enriched syngas 136 to achieve the effective H₂/CO ratio based on mass balance simulations without undue experimentation.

In some preferred embodiments of FIGS. 1-3, the processes are integrated such that the syngas production unit 100, GTL system 106, utilities generation unit 120, synthetic product hydroprocessing system 200, SMR unit 300, or a combination thereof, are located in close proximity. This close proximity allows the processes to transfer the streams described above between units, typically by conduit or pipeline, such that there is no transferring of the intermediate product via transportation vehicles. Some alternate embodiments may include intermediate storage (not shown) to provide maximum efficiency and independent start-up and operation of the various units. Furthermore, in some embodiments, some of the methane-containing feedstock 102 is used as required for make-up fuel to the utilities generation unit 120.

The advantage of the current invention is that the loss of CO and H₂ in the overall GTL process is effectively minimized while any other hydrocarbon and other gases in the F-T reactor off-gas are utilized as fuel for power or steam generation. When CO₂ from upstream natural gas, as well as from raw syngas effluent of the syngas generation units are removed and recycled to a syngas generator, such as a SMR, additional CO is generated and steam demand is reduced. Integration of a methane-containing feedstock, a GTL off-gas, and a hydroprocessor off-gas further reduce the number of unit operations and minimize loss of valuable feedstock.

Additional advantages include:
No need for compression of feed streams both to the off-gas membrane and to the CO recovery unit;
Required compression is limited to the pure H₂ and CO streams that are small in volume;
No pretreatment is required for both membrane and CO recovery unit. (e.g., removal of CO₂, moisture, etc. would be required if a cryogenic unit is used);
Meet the key process requirements: CO and H₂ recovered; N₂, C₁-C₅ hydrocarbons, Ar, CO₂ rejected from the F-T loop, and rejected “inert gases” used as fuel in the utilities unit; and
Integration with a hydroprocessor system eliminates a separate purge H₂ recovery stage, as well as CO₂ removal and utilization.

Although the present invention has been described in considerable detail with reference to certain preferred versions thereof, other versions are possible. For example, where process streams are combined, the combination can occur in specific equipment shown in preferred embodiments, or in piping, or in other process equipment not shown herein.

Furthermore, separation membrane devices, hydrocarbon synthesis units and other units described herein may vary in construction. For example, one hydroprocessing system may use equipment referred to as hydrocracker, whereas another may use a hydrotreator to effect the desired product production. There is also a variety of devices known in the art to construct and control the described devices. Therefore, the spirit and scope of the appended claims should not be limited to the description of the preferred versions contained herein.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A process for integrating a syngas production unit, a GTL system, and a utilities generation unit, the process comprising the steps of:
(a) providing an integrated processing system comprising:
(i) a syngas production unit;
(ii) a GTL system;
(iii) a utilities generation unit;
(iv) an off-gas membrane separator; and
(v) a CO recovery unit,
(b) supplying a methane-containing feedstock comprising methane,
(c) forming a syngas;
(d) forming a GTL off-gas;
(e) separating a first portion of GTL off-gas in said off-gas membrane separator to form an H₂-enriched gas and an H₂-lean/CO-rich gas;
(f) combining a CO-enriched syngas and said H₂-enriched gas to form an H₂-enriched syngas;
(g) producing a synthetic hydrocarbon product is said GTL system;
(h) feeding said H₂-lean/CO-rich gas to said CO recovery unit to form a CO enriched gas and a combustible tail gas;
(i) combining said CO-enriched gas and said syngas to form said CO enriched syngas;
(j) feeding said combustible tail gas to said utilities generation unit; and
(k) producing a utility product in said utilities generation unit.

2. The process of claim 1, further comprising the step of combining a second portion of GTL off-gas with said H₂-enriched syngas to form a GTL feedstock, wherein said GTL feedstock is formed with an effective H₂/CO ratio for the production of synthetic hydrocarbon products.

3. The process of claim 2, further comprising the step of recycling a third portion of GTL off-gas to said syngas production unit.

4. The process of claim 3, further comprising the step of feeding a fourth portion of GTL off-gas to said utilities generation unit.

5. The process of claim 1, wherein said step of forming a syngas further comprises the steps of:
(a) separating said methane-containing feedstock into a CO₂-enriched feedstock and a CO₂-lean feedstock in a feedstock membrane separator;
(b) feeding said CO₂-lean feedstock to said syngas production unit to form an ATR/POX syngas;
(c) feeding said CO₂-enriched feedstock to a SMR unit to form a SMR syngas; and
(d) combining said ATR/POX syngas and said SMR syngas to form said syngas.

6. The process of claim 5, further comprising the step of recycling a third portion of GTL off-gas to said SMR unit.

7. The process of claim 6, further comprising the step of feeding a fourth portion of GTL off-gas to said utilities generation unit.

8. A process for integrating a syngas production unit, a GTL system, a synthetic product hydrocracking system, and a utilities generation unit, the process comprising the steps of:
(a) providing an integrated processing system comprising:
(i) a syngas production unit;
(ii) a GTL system;
(iii) a utilities generation unit;
(iv) an off-gas membrane separator;
(v) a CO recovery unit;
(vi) a syngas membrane separator;
(vii) an H₂ PSA unit; and
(viii) a synthetic product hydroprocessing system;
(b) supplying a methane-containing feedstock comprising methane,
(c) forming a syngas;
(d) forming a GTL off-gas;
(e) separating a first portion of GTL off-gas and a second portion of hydroprocessor purge in said off-gas membrane separator to form an H₂-enriched gas and an H₂-lean/CO-rich gas stream;
(f) combining a CO-enriched syngas and a first portion of H₂-enriched gas to form an H₂-enriched syngas;
(g) producing a synthetic hydrocarbon product is said GTL system;
(h) feeding said H₂-lean/CO-rich gas to said CO recovery unit;
(i) obtaining a CO enriched gas and a combustible tail gas from said CO recovery unit;
(j) feeding said combustible tail gas to said utilities generation unit;
(k) producing a utility product in said utilities generation unit;
(l) combining a second portion of GTL off-gas with said H₂-enriched syngas to form a GTL feedstock, wherein said GTL feedstock is formed with an effective H₂/CO ratio for the production of a synthetic hydrocarbon product;
(m) separating a first portion of syngas in said syngas membrane separator to form an H₂-enriched syngas and an H₂-lean syngas;
(n) combining said syngas, said CO-enriched gas, and said H₂-lean syngas to form said CO enriched syngas;
(o) feeding a second portion of said H₂-enriched gas and said H₂-enriched syngas to said H₂ PSA unit to form a high purity H₂ and a H₂ PSA tail gas;
(p) combining a first portion of hydroprocessor purge and said high purity H₂ to form a hydrocracker H₂ feed;
(q) feeding said synthetic hydrocarbon product and said hydrocracker H₂ feed to said synthetic product hydrocracking system to form a hydrocracker product and a hydrocracker purge stream; and
(r) dividing said hydrocracker purge stream to form said first portion of hydroprocessor purge and said second portion of hydroprocessor purge.

9. The process of claim 8, further comprising the step of recycling a third portion of GTL off-gas and said H₂ PSA tail gas to said syngas production unit.

10. The process of claim 9, further comprising the step of feeding a fourth portion of GTL off-gas to said utilities generation unit.
